(19) United States
(12) Patent Application Publication
(10) Pub. No.: US 2011/0129541 A1
(43) Pub. Date: Jun. 2, 2011

(54) SUSPENSION DELIVERY SYSTEM FOR THE SUSTAINED AND CONTROLLED LOCAL RELEASE OF PHARMACEUTICALS

(75) Inventors: Jianbing Chen, Wayland, MA (US); Hong Guo, Wayland, MA (US); Paul Ashton, Newton, MA (US)

(73) Assignee: Control Delivery Systems, Inc., Watertown, MA (US)

(21) Appl. No.: 12/870,618

(22) Filed: Aug. 27, 2010

Related U.S. Application Data
(63) Continuation of application No. 10/972,750, filed on Oct. 25, 2004, now abandoned.
(60) Provisional application No. 60/514,864, filed on Oct. 27, 2003.

(51) Int. Cl. A61K 31/485; A61K 9/10; A61P 29/00; A61P 25/04

(52) U.S. Cl. 424/499; 424/501; 514/282

(57) ABSTRACT

The invention relates to a novel suspension delivery system for the sustained and controlled release of pharmaceuticals. Methods of preparation and use are also disclosed.

### RELATED APPLICATIONS

This application is a continuation of U.S. patent application Ser. No. 10/972,750, filed Oct. 25, 2004, which claims the benefit of U.S. Provisional Application No. 60/514,864, filed Oct. 27, 2003, the specifications of which are incorporated by reference herein in their entirety.

### BACKGROUND OF THE INVENTION

The effect of an agent in treating a physiological condition is often determined by the physical characteristics of the formulation used to deliver the agent and the mode of delivering the formulation. Commonly used formulations combine a super disintegrant with one or more drugs in the form of a capsule, tablet, pellet, or granule for oral delivery of such drug or drugs. For example, U.S. Pat. Nos. 6,555,133 and 6,596,311 describe the use of super disintegrants to form tablets and capsules for the delivery of pharmaceutical agents. The capsules and tablets of these patents and other literature are typically administered orally in solid dosage forms. However, such formulations often lead to premature decomposition in the harsh environment of the digestive tract and unwanted side effects, particularly as a result of their oral modes of administration and their inevitable widespread circulation throughout the body.

Other formulations provide a drug in a suspension form to a patient and are conveniently capable of localized administration by injection. However, such formulations often do not provide sustained release of the drug for a long period of time and often result in undesired phase separation as a result of the immiscibility of the formulation's individual components, or as a result of prolonged contact of the formulation with the suspension medium during long-term storage.

What is needed is an improved formulation and drug delivery system that provides for localized, sustained- and controlled-release drug delivery.

### SUMMARY OF THE INVENTION

The present invention provides for an improved drug delivery system for achieving localized, sustained- and controlled-release drug delivery. In particular, one or more agents are combined with one or more disintegrants to form one or more pellets, which may be combined with an aqueous medium and administered as an injectable (or otherwise administrable) suspension. In some embodiments, the disintegrant is a super disintegrant, such as those described in U.S. Pat. Nos. 6,555,133, and 6,596,311. In some embodiments, the agents are pharmaceutically acceptable salts or prodrugs thereof. In some embodiments, the agents are codrugs, or pharmaceutically acceptable salts or prodrugs thereof. The invention also provides a method of preparing the drug delivery system of the present invention. The invention also provides a method of treating a patient in need of treatment, comprising administering to such patient one or more agents via the drug delivery system of the present invention. The invention also provides a kit containing the inventive system, as well as a method of manufacturing and providing such kit.

### BRIEF DESCRIPTION OF THE FIGURES

FIG. 1 shows comparative in-vitro release profiles of pellets having 10% and 5% concentrations of disintegrant compound Ac-Di-Sol (ADS).

FIG. 2 shows comparative in-vitro release profiles of pellets having 10% and 5% concentrations of disintegrant compound sodium starch glycolate (SSG).

FIG. 3 shows comparative morphine release profiles of pellets having 10% ADS and 10% SSG.

FIG. 4 shows comparative morphine release profiles of pellets having 5% ADS and 5% SSG.

FIG. 5 shows comparative morphine release profiles for In/Ex pellets (pellets containing 5% ADS added before granulation and 5% ADS mixed with dried granules) and In pellets (pellets containing 10% ADS added before granulation).

FIG. 6 shows comparative morphine release profiles for pellets containing 5% ADS in 1% hyaluronic acid and in 0.1M phosphate buffer saline, pH 7.4.

FIG. 7 shows comparative morphine release profiles for In/Ex pellets (containing 5% ADS added before granulation and 5% ADS mixed with dried granules) in 1% hyaluronic acid and in 0.1M phosphate buffer saline, pH 7.4.

FIG. 8 shows comparative morphine release profiles for pellets containing 10% ADS added before granulation in 1% hyaluronic acid and in 0.1M phosphate buffer saline, pH 7.4.

### DETAILED DESCRIPTION OF THE INVENTION

The present invention provides an improved drug delivery system comprising a pellet that is prepared by combining one or more agents and one or more disintegrant compounds. The pellet is then dispersed in aqueous medium to form a suspension, and the suspension then is administered to a patient. In preferred embodiments, the suspension is formed immediately prior to administration. In preferred embodiments, the suspension is administered by injection, although other modes may be used (e.g., administration via catheter).

Disintegrant compounds facilitate the disintegration of dry drug dosage forms (e.g., tablets, capsules, granules, etc.) in aqueous solutions. Super disintegrants are disintegration agents that can be used in a fractional amount of normal disintegrants to obtain the same effect. In certain embodiments, at least one disintegrant compound is used. In certain embodiments at least one disintegrant compound is a super disintegrant.

Exemplary disintegrant compounds may include starch, microcrystalline cellulose, formaldehyde casein, alginic acid and numerous other compounds. Many disintegrants useful with the invention are super disintegrants. Examples of super disintegrants include, without limitation, crospovidone (e.g., Kollidon® CL), carmellose, directly compressible starches (e.g., Starch 1500®), modified starches (e.g., carboxymethyl starch, sodium starch glycolate), natural starches (e.g., maize starch, potato starch), cross-linked polyvinyl pyrrolidone, modified celluloses (e.g., cross-linked sodium carboxymethylcellulose (e.g. croscarmellose, Ac-Di-Sol®)), and combinations, complexes and salts of any of the foregoing.

Examples of pellets formed and used with the invention may include those widely known in the art. For example, U.S. Pat. Nos. 6,555,133 and 6,596,311 describe exemplary pellets and capsules that combine super disintegrants with drugs to form drug formulations. The teachings of both patents are incorporated herein by reference in their entirety.

As noted above, super disintegrants are effective in relatively small amounts. Therefore, in certain embodiments, the inventive system has at least one super disintegrant with a mass of up to about 30% of the entire pellet. In certain embodiments, the mass is up to about 15%, or even up to about 5% or less. In certain embodiments, the super disintegrants increase the aqueous disintegration rate of the pellet by about 15% or more, by greater than about 30%, or even by greater than about 50%, as compared to such rate of when no super disintegrant is used.

Furthermore, the pellet may be formed in various sizes. In certain embodiments, the pellet may be up to about 0.5 mm in diameter, or up to about 5 mm or even over 10 mm in diameter. The pellet may be up to about 1 mm in height, or even up to about 10 mm. It may take on variable weights. For example, the pellet may be up to about 1 mg, or up to about 10 mg, or even up to about 250 mg. In certain embodiments, the pellet is between about 1.5 mm and about 2.5 mm in diameter, between about 1.0 mm and 1.5 mm in height, and weighs about 5 mg.

In certain embodiments, the pellet may include, in addition to one or more disintegrant compounds, a pharmaceutically acceptable carrier, such as, but not limited to, magnesium stearate. Such additional pharmaceutically acceptable carriers may be present in amounts up to about 0.5% of the total mass of the pellet, or even up to about 10% or greater depending on the nature of the carrier.

In certain embodiments, the pellet is not coated or in capsule form.

In certain embodiments, the aqueous medium may (but need not) include a biocompatible buffer or gel, such as, but not limited to, hyaluronic acid (HA) or physiological saline. In certain embodiments, the buffer contains up to about 5% HA. In other embodiments, the buffer may contain up to about 2% or up to about 1% of HA.

The invention may be used to provide sustained- and controlled-release drug delivery through the convenience of injection. In certain embodiments, agents prepared according to the invention exhibit comparable physiological release profiles to those delivered through solid dosage form.

In certain embodiments, the invention may provide controlled release of an agent over an extended period. In certain embodiments, the controlled release occurs over a period of at least 24 hours; preferably, the controlled release occurs over at least 2 days, or even at least one or two weeks or at least one month.

In certain embodiments, the invention allows for the injectable (and therefore more efficient) delivery of agents that ordinarily are not readily deliverable in the form of a suspension (e.g., peptides, proteins, steroids, and non-steroidal anti-inflammatory drugs) while avoiding degradation of such agents in the aqueous component of the suspension. In preferred embodiments, the suspension is formed immediately prior to administration to a patient by combining a pellet containing at least one agent and at least one super disintegrant with an aqueous medium and gently shaking until a suspension forms. The pellet approach thereby reduces the time the suspension exists ex vivo and, as a result, the degradation of the at least one agent contained in the suspension. In certain embodiments, agents delivered with such suspensions are at least 10% more stable than when delivered in the form of a suspension prepared other than according to the present invention. In certain embodiments the increased stability is at least 25%, or even 50% or more.

The present invention may reduce or eliminate the need for other materials normally used to increase the stability of a suspension. These include but are not limited to surfactants, anti-oxidants and preservatives. The present invention is also suitable for delivering agents that are sensitive to storage in the presence of water. Such water-sensitive agents may include, for example, compounds that undergo hydrolysis or otherwise decompose in aqueous solution. As an exemplary (but not limiting) embodiment, the agent is a codrug (e.g., triamcinolone acetonide (TA) and bis (hydroxymethyl)-5-fluorouracil (5FU)) that hydrolyzes to liberate in aqueous solution to form the individual constituents. The invention allows for the storage of such agents without the accompanying hydrolysis, while still allowing for the regeneration and administration of the agent when needed. The agent may hydrolyze or otherwise decompose in aqueous medium at a rate corresponding to a half-life of about one week or more at pH 7.4 and 25° C. In other embodiments, such half-life is less than one week, while in other embodiments the half-life is less than about 48 hours. In still other embodiments, the agent has a decomposition half-life of less than about 24 hours or even less than about 6 hours. In other embodiments, the half-life is less than about 1 hour, while in other embodiments the half-life is less than about 10 minutes.

Those skilled in the art will readily recognize that the invention may be applied to any agent that is subject to hydrolysis in aqueous solution, irrespective of the hydrolysis rate. For example, but certainly without limitation, suitable agents may include esters (e.g., aspirin), thiol esters (e.g., spironolactone), amids (e.g., chloramphenicol), imides (e.g., phenobarbitone), lactams (e.g., methicillin), lactones (e.g., spironolactone), etc.

As discussed more thoroughly below, in certain embodiments, the agent may include one or more codrugs. In particular embodiments, the agent includes at least one codrug having first and/or second molecule residues, or a bond linking the molecule residues, that are/is unstable in aqueous solution. In certain embodiments, a codrug residue prepared and delivered according to the invention has a decomposition half-life that is at least 10% longer than when prepared and stored in aqueous solution; in certain embodiments the half-life is at least 25% longer, or even 50% longer or more.

In another aspect, the invention allows for the long-term storage of one or more agents in the form of a pellet and the ready reconstitution of the suspension in aqueous medium when needed. In certain embodiments, agents that are unstable in aqueous solution may be processed and stored in the form of a pellet but are made readily available for injection or other administration after suspension in aqueous medium according to the invention.

Storage in pellet form may also reduce the need to use preservatives and/or anti-oxidants to maintain the long-term stability of the agent, as compared to systems that store the agent in aqueous medium. In certain embodiments, the agent is stored in pellet form with the disintegrant compound but without the use of a preservative. In such embodiments, the decomposition half-life of the agent as a result bacterial growth on the pellet may be similar to the decomposition of the agent when stored in aqueous medium using a preservative.

Storing the agent in pellet form may also reduce, and in some cases eliminate, the need to use an anti-oxidant. In certain embodiments, the at least one agent stored in pellet form without an anti-oxidant may oxidize at a rate that is similar to the oxidation rate of the agent when stored with an anti-oxidant in an aqueous medium.

The pellet and the aqueous medium may be sterilized prior to combining them for administration. In certain embodiments, sterilizing the pellet prior to combining in aqueous medium may result in slower or less drastic decomposition of the agent than when sterilization occurs after the suspension is formed. In certain embodiments, sterilizing the pellet prior to combining in aqueous medium results in decomposition of the agent that is at least 10% or even 40% lower than when sterilizing a suspension containing the agent and the aqueous medium together. Sterilizing the pellet and the aqueous medium prior to combining them may allow the practitioner to avoid applying filtration, radiation, chemical and other potentially destructive sterilization processes to the formulation.

The use of disintegrant compounds may also reduce the need to use surfactants to facilitate the release of the agent. In certain embodiments, the disintegrant is used in lieu of a surfactant. In certain embodiments, the pellet includes at least one agent and at least one disintegrant compound, the at least one agent having a release rate that is similar to the release rate of a baseline suspension containing the at least one agent and a surfactant. In certain embodiments, the release rate of the pellet containing the disintegrant compound is within about 1% of the release rate of the baseline suspension.

As discussed more fully below, the invention also provides for the localized delivery of agents that are otherwise insufficiently soluble in aqueous medium and are, as a result, ordinarily unsuited to delivery by injection. Such agents are typically administered orally in solid dosage forms, which results in the agents being delivered systemically, often with undesired degradation prior to reaching the intended site and with unwanted side effects. For example, the invention enables the injectable delivery of agents that have low solubility or less. In preferred embodiments, the systemic concentration of the locally delivered agent(s) remains low, preferably at levels that are insufficient to provide a therapeutic effect. In certain embodiments, the serum concentration of the agent is less than about 50% of the serum concentration required to produce a therapeutic effect. In other embodiments, the serum concentration of the agent is less than about 30%, or even less than about 10% of the serum concentration required to produce a therapeutic effect.

In preferred embodiments, the inventive system is substantially pyrogen-free.

Those skilled in the art will recognize that numerous drugs may be used with the invention. In certain embodiments according to the present invention, at least one of the agents is an antineoplastic; an antibacterial; a non-steroidal anti-inflammatory drug (NSAID); a steroid; a glucocorticoid or other anti-inflammatory corticosteroid, such as a topical anti-inflammatory steroid; an anti-angiogenesis agent; an alkaloid analgesic, such as an opioid analgesic; an antiviral, such as a nucleoside antiviral or a non-nucleoside antiviral; an anti-benign prostatic hypertrophy (BPH) agent; an antibiotic compound; an anti-fungal compound; an antiproliferative compound; an anti-glaucoma compound; an immunomodulatory compound; a cell transport/mobility impeding agent; a cytokine; a peptide; a protein; a pegylated agent; an alpha-blocker; an anti-androgen; an anti-cholinergic agent; an adrenergic agent; a purinergic agent; a dopaminergic agent; a local anesthetic; a vanilloid; a nitrous oxide inhibitor; an anti-apoptotic agent; a macrophage activation inhibitor; an antimetabolite; a neuroprotectant; a calcium channel blocker; a gamma-aminobutyric acid (GABA) antagonist; an alpha agonist; an anti-psychotic agent; a tyrosine kinase inhibitor; a nucleoside compound; a nucleotide compound; another therapeutic compounds; and analogs, derivatives, pharmaceutically acceptable salts, esters, prodrugs, codrugs, and protected forms thereof.

Suitable NSAIDs include diclofenac, etoldolac, fenoprofen, floctafenine, flurbiprofen, ibuprofen, indoprofen, ketoprofen, ketorolac, lornoxicam, morazone, naproxen, perisoxal, pirprofen, pranoprofen, suprofen, suxibuzone, tropesin, ximoprofen, zaltoprofen, zileuton, and zomepirac, and analogs, derivatives, pharmaceutically acceptable salts, esters, prodrugs, codrugs, and protected forms thereof.

Suitable alkaloid analgesics include desmorphine, dezocine, dihydromorphine, dimepbeptanol, eptazocine, ethylmorphine, glafenine, hydromorphone, isoladol, ketobenidone, p-lactophetide, levorphanol, moptazinol, metazocin, metopon, morphine, nalbuphine, nalmefene, nalorphine, naloxone, norlevorphanol, normorphine, oxmorphone, pentazocine, phenperidine, phenylramidol, tramadol, and viminol, and analogs, derivatives, pharmaceutically acceptable salts, esters, prodrugs, codrugs, and protected forms thereof.

Suitable glucocorticoids include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, diflorasone, diflucortolone, difuprednate, enoxolone, fluazacort, flucloronide, flumethasone, flunisolide, fluocinolone acetonide, fluocinonide, flucloronide, flumethasone, flunisolide, fluocortin butyl, fluocortolone, fluorometholone, fluperolone acetate, fluprednisolone, flurandrenolide, fluticasone propionate, hydrocortamate, hydrocortisone, meprednisone, methylprednisolone, paramethasone, prednisolone, prednisolone 21-diethylaminoacetate, fluprednidene acetate, formocortal, loteprednol etabonate, medrysone, mometasone furoate, prednicarbate, prednisolone, prednisolone 25-diethylaminoacetate, prednisolone sodium phosphate, prednisone, prednival, prednylidene, triamcinolone, triamcinolone acetonide, triamcinolone benetonide, and triamcinolone hexacetonide, and analogs, derivatives, pharmaceutically acceptable salts, esters, prodrugs, codrugs, and protected forms thereof.

Other suitable steroids include halcinonide, halbetasol propionate, halometasone, halopredone acetate, hormaone, isoflupredone, loteprednol etabonate, mazipredone, rimexolone, and tixocortol, and analogs, derivatives, pharmaceutically acceptable salts, esters, prodrugs, codrugs, and protected forms thereof.

Suitable BPH drugs include finasteride and osaterone, and analogs, derivatives, pharmaceutically acceptable salts, esters, prodrugs, codrugs, and protected forms thereof.

Suitable antineoplastic compounds include alitretinoin (9-cis-retinoic acid); bleomycins, including bleomycin A; capecitabine (5′-deoxy-5-fluoro-cytidine); carubicin; chlorozotocin, chromomycins, including chromomycin A₃, cladribine; colchicine, cytarabine; daunorubicin; demecolcine, denopterin, docetaxel, doxyifluridine, doxorubicin; dromostanolone, edatrexate, enocitabine, epirubicin, epitiostanol, estramustine; etop( ) side; floxuridine, fludarabine, 5-fluorouracil, formestane, gemcitabine; irinotecan; lentinan, lonidamine, melengestrol, melphalan; menogaril, methotrexate; mitolactol; nogalamycin; nordihydroguaiaretic acid, olivomycins such as olivomycin A, paclitaxel; pentostatin; pirarubicin, plicamycin, porfiromycin, prednimustine, puromycin; ranimustine, ristocetins such as ristocetin A; temozolamide; teniposide; tomudex; topotecan; tubercidin, ubenimax, valrubicin (N-trifluoroacetyladriamycin-14-valerate), vinorelbine, vinblastine, vindesine, vinorelbine, and zorubicin and analogs, derivatives, pharmaceutically acceptable salts, esters, prodrugs, codrugs, and protected forms thereof.

Suitable antibacterial compounds include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefinenoxime, cefinetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cepholosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A₁, lincomycin, lomefloxacin, loracarbef, lymecycline, meropenam, metampicillin, methacycline, methicillin, mezlocillin, micronaomicin, midecamycins such as midecamycin A₁, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin O, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, codrugs, and protected forms thereof.

In some embodiments, the agent is a codrug, or a prodrug or pharmaceutically acceptable salt thereof. In certain of such embodiments, the first residue is selected from an antineoplastic; an antibacterial; an NSAID; steroid; a glucocorticoid or other anti-inflammatory corticosteroid, such as a topical anti-inflammatory steroid; an anti-angiogenesis agent; an alkaloid analgesic, such as an opioid analgesic; an antiviral, such as a nucleoside antiviral or a non-nucleoside antiviral; a BPH compound; an antibiotic compound; an anti-fungal compound; an antiproliferative compound; an anti-glaucoma compound; an immunomodulatory compound; a cell transport/mobility impeding agent; a cytokine; a peptide; a protein; a pegylated agent; an alpha-blocker; an anti-androgen; an anti-cholinergic agent; an adrenergic agent; a purinergic agent; a dopaminergic agent; a local anesthetic; a vanilloid; a nitrous oxide inhibitor; an anti-apoptotic agent; a macrophage activation inhibitor; an antimetabolite compound; a neuroprotectant; a calcium channel blocker; a GABA acid antagonist; an alpha agonist; an anti-psychotic compound; a tyrosine kinase inhibitor; a nucleoside compound; a nucleotide compound; another therapeutic compounds; and analogs, derivatives, pharmaceutically acceptable salts, esters, prodrugs, codrugs, and protected forms thereof.

In certain of such embodiments, the second residue is selected from an antineoplastic; an antibacterial; an NSAID; steroid; a glucocorticoid or other anti-inflammatory corticosteroid, such as a topical anti-inflammatory steroid; an anti-angiogenesis agent; an alkaloid analgesic, such as an opioid analgesic; an antiviral, such as a nucleoside antiviral or a non-nucleoside antiviral; a BPH compound; an antibiotic compound; an anti-fungal compound; an antiproliferative compound; an anti-glaucoma compound; an immunomodulatory compound; a cell transport/mobility impeding agent; a cytokine; a peptide; a protein; a pegylated agent; an alpha-blocker; an anti-androgen; an anti-cholinergic agent; an adrenergic agent; a purinergic agent; a dopaminergic agent; a local anesthetic; a vanilloid; a nitrous oxide inhibitor; an anti-apoptotic agent; a macrophage activation inhibitor; an antimetabolite compound; a neuroprotectant; a calcium channel blocker; a GABA acid antagonist; an alpha agonist; an anti-psychotic compound; a tyrosine kinase inhibitor; a nucleoside compound; a nucleotide compound; another therapeutic compounds; and analogs, derivatives, pharmaceutically acceptable salts, esters, prodrugs, codrugs, and protected forms thereof.

In certain codrug embodiments, the first residue is an NSAID compound. In some embodiments, the second residue is an analgesic compound. In certain embodiments, the first residue is diclofenac or ketorolac and the second residue is morphine.

In another aspect, the invention contemplates administering agents to a patient. The inventive system facilitates this administration by enabling the localized delivery of agents, preferably by injection. Certain agents are ordinarily delivered by solid dosage form because of their poor solubility in aqueous medium. The inventive system provides that such agents may be injected at or near the locus of desired therapeutic activity, where they will be released slowly into the surrounding tissue to achieve sustained release, thereby producing a high and prolonged local concentration of the agent(s). Because systemic administration is avoided by this method, the systemic concentrations of the agents may remain low, while the localized concentrations may be maintained within a desired therapeutic range over a period of time ranging from days to months.

In other embodiments, the invention contemplates manufacturing and providing a kit containing the inventive system. In certain embodiments, the kit may include a pellet or pellets comprising at least one agent in combination with at least one disintegrant compound as described above. The kit also may include a vial, the vial being suitable for use in suspending the pellet in aqueous medium. The pellet may be provided in the vial, or separately. When the pellet is provided in the vial, the vial may, optionally, be sealed. In certain embodiments, the kit also may include an aqueous medium in which the pellet may be suspended. In certain embodiments in which the pellet is not provided in the vial, the medium may be optionally provided in the vial or separately. In certain embodiments, the medium is provided in a syringe that allows the injection of the medium into the pellet-containing vial. In still other embodiments, the medium is provided in a vial with a removable cap, and the pellet is provided separately. The suspension is formed by removing the cap, inserting the pellet into the vial, replacing the cap and gently shaking the vial. The suspension then is administered as needed to a patient. In the foregoing and other embodiments, the medium and pellet may be shaken to form the suspension which is subsequently drawn by a syringe and administered as needed to a patient.

In certain embodiments, the kit may also include instructions for suspending the pellet in aqueous medium. In certain embodiments, the kit includes instructions for preparing suspensions having concentrations suitable for providing specified doses of an agent as needed by a patient. In certain embodiments the kit may also include instructions for administering a suspension according to the invention.

An exemplary, but non-limiting, embodiment may include: a pellet with 90-95% morphine-diclofenac-maleate codrug and 5-10% superdisintegrant (e.g., sodium starch glycolate, croscarmellose sodium). The pellet may also contain 0.2-0.4% magnesium stearate. The pellet is between 1.5 mm and 2.5 mm in diameter, between about 1.0 mm and 1.5 mm in height, and weighs about 5 mg. An aqueous medium is used and may optionally include hyaluronic acid (HA). Upon combining the pellet with aqueous medium, a suspension forms and is administered to a patient by injection.

One of skill in the art may further comprehend the invention by reference to the following non-limiting examples.

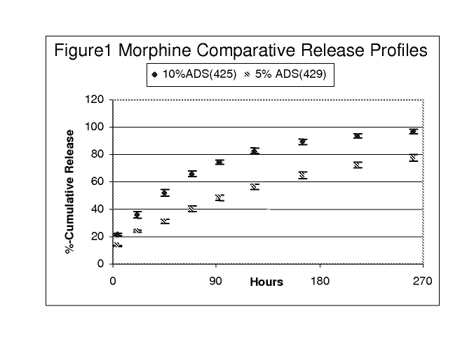

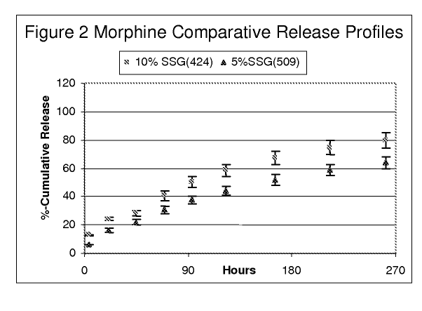

FIGS. 1 and 2 show the comparative in-vitro release profiles of pellets with different concentration (10% and 5%) of Ac-Di-Sol (ADS, FIG. 1) and sodium starch glycolate (SSG, FIG. 2). The profiles indicate that morphine release rate increases with higher concentration of ADS or SSG in the pellet formulation, illustrating the ability to control the release rate of the agent by use of disintegrant compounds.

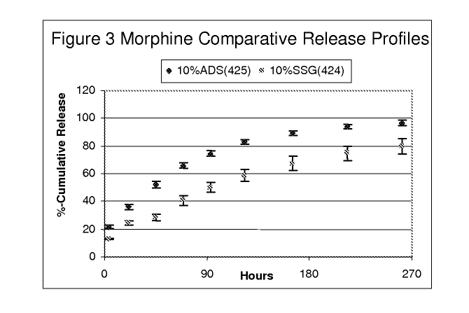

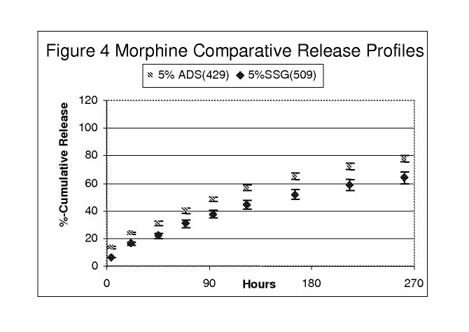

FIGS. 3 and 4 show the morphine release profiles, comparing the effect of ADS and SSG having concentrations of 10% (FIG. 3) and 5% (FIG. 4). For both concentrations, pellets with ADS release more morphine over the measured period, further illustrating the ability to use the invention to control the delivery of an agent.

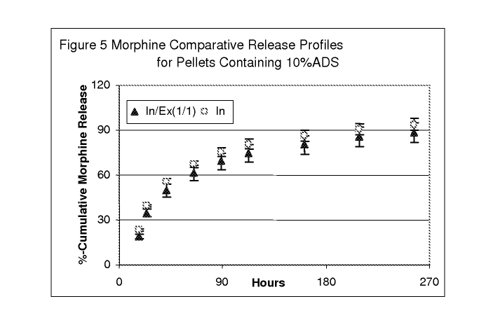

FIG. 5 shows the comparative morphine release profiles for two batches of pellets: In/Ex (1/1) containing 5% ADS added before granulation and 5% ADS mixed with the dried granules, and In containing 10% ADS added before granulation. “In” refers to “intragranularly,” meaning the disintegrant is incorporated prior to granulation, and “Ex” refers to “extra-granularly,” meaning the disintegrant is added after granulation.

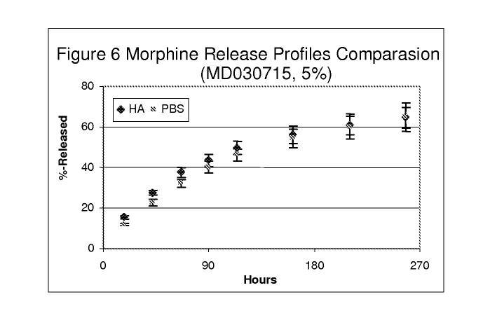

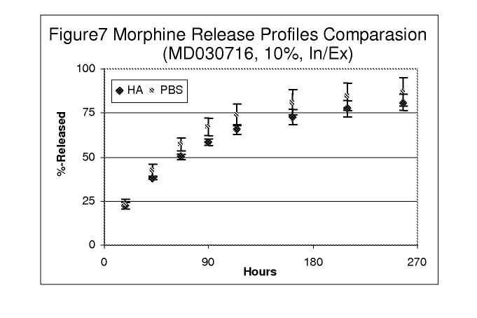

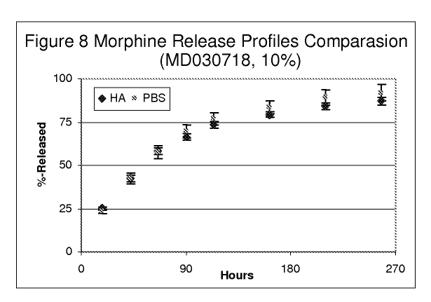

FIG. 6, FIG. 7, and FIG. 8 show the comparative morphine release profiles in different dispersing media (1% aqueous hyaluronic acid (HA) or 0.1M of phosphate buffer saline (PB), pH 7.4) for three batches of pellets: 5% ADS (FIG. 6), 5% ADS added before granulation and 5% ADS mixed with the dried granules (FIG. 7), and 10% ADS added before granulation (FIG. 8). No significant difference in release profile was observed for all three batches, suggesting HA in the dispersing medium did not effect the release rates.

### DEFINITIONS

The term “active” as used herein means pharmacologically, biologically, or pharmaceutically active.

The term “administer” means insert, inject, implant, or deliver in any other fashion. In preferred embodiments at least one agent is administered according to the invention by injection. Agents may also be administered by catheter.

The terms “agent” and “drug” as used herein are synonymous with each other and with “at least one agent,” “at least one drug,” “compound,” and “at least one compound,” and mean (a) a physiologically active entity, or a prodrug or pharmaceutically acceptable salt thereof, or (b) a codrug, or a prodrug or pharmaceutically acceptable salt thereof. In still other embodiments, the terms “agent” and “drugs” refer to a plurality of drugs, proteins, peptides, etc. In certain embodiments, the agent may be in granular form, powdered form, nanoparticle form, or microsphere form.

The term “codrug” as used herein means a compound comprising a first molecule residue associated with a second molecule residue, wherein each residue, in its separate form (e.g., in the absence of the association), is an active agent or a prodrug of an active agent. In preferred embodiments, either one or both of the first and second molecule residues are small molecules. The association between said residues can be either ionic or covalent and, in the case of covalent associations, either direct or indirect through a linker. The first molecule can be the same or different from the second. Exemplary formulae for codrugs are set forth in formulae I, Ia, II, IIa, III, IIIa, and IV:

A₁*(−L−A₂*)ₙ (I)

A₁*(−A₂*)ₙ (Ia)

A₁*−L−A₂* (II)

A₁*−A₂* (IIa)

A₂*−L−A₁*−L−A₂* (III)

A₂*−A₁*−A₂* (IIIa)

A₁*::A₂* (IV)

wherein each of A₁*, A₂*, and L are defined as follows:

A₁* is a residue of a first active compound, A₁;

A₂* is a residue of a second active compound, A₂, which may be the same as or different from A₁;

L is a linking group selected from a direct bond and a divalent organic linking group;

n is an integer having a value of from 1 to 4, preferably 1;

and :: is an ionic bond.

Codrugs, as that term is used herein, are more fully described in U.S. Pat. No. 6,051,576, the disclosure of which is incorporated herein in its entirety.

The term “covalently linked” as used herein means either a direct covalent bond between two species, or an indirect association where two species are not directly bonded but are both covalently bonded to an intermediate linker

An “effective” amount of an agent, with respect to methods of treatment, is synonymous with a “therapeutically effective amount” or “effective dosage,” and refers to an amount of the agent in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose.

In general, “low solubility” means that the agent is only very slightly soluble in an aqueous medium (e.g., aqueous solutions having pH in the range of about 5 to about 8, and in particular to physiologic solutions, such as blood, blood plasma, etc.). Some agents, e.g., low-solubility agents, will have solubilities of less than about 1 mg/ml in the medium, less than about 100 μg/ml, preferably less than about 20 μg/ml, more preferably less than about 15 μg/ml, and even more preferably less than about 10 μg/ml. Solubility in water is measured at a temperature of 25° C. as measured by the procedures set forth in the 1995 USP, unless otherwise stated. According to the invention, compounds which are soluble (greater than about 100 mg/ml), moderately soluble (about 100 mg/ml to about 10 mg/ml), slightly soluble (about 10 mg/ml to about 1 mg/ml), very slightly soluble (about 1 mg/ml to about 0.1 mg/ml) and practically insoluble or insoluble compounds (less than about 0.1 mg/ml, preferably less than about 0.01 mg/ml) are contemplated.

A “patient” to be treated by the inventive system refers to either a human or non-human animal.

A “pellet,” as used herein, means a capsule, tablet, granule, particle, or any other solid form.

The phrase “pharmaceutically acceptable carrier” as used herein means a pharmaceutically acceptable material, composition or vehicle, such as a liquid or solid filter, diluent, adjuvant, excipient, solvent or encapsulating material, suitable for carrying or transporting a subject agent within the body. In certain embodiments the transportation may occur between organs, tissues or cells, or between a tissue and a cell, or between an organ and any of the foregoing. Each carrier must be “acceptable” in the sense of being compatible with other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations, such as magnesium stearate. Preferred carriers are non-pyrogenic, i.e., do not substantially elevate the body temperature of a patient receiving the formulation.

“Physiological conditions” describe the conditions inside an organism, i.e., in vivo. Physiological conditions include the acidic and basic environments of body cavities and organs, enzymatic cleavage, metabolism, and other biological processes, and preferably refer to physiological conditions in a vertebrate, such as a mammal.

The term “prodrug” as used herein means a first residue associated with a second residue, wherein one of the residues is active and one is not active. In preferred embodiments, either one or both of the first and second residues are small molecules. In some embodiments, the prodrug may be biologically inactive in its prodrug form. The association between said residues is covalent and can be either direct or indirect through a linker. Prodrugs of active compounds include esters, as well as anhydrides, amides, and carbamates that are hydrolyzed in biological fluids to produce the parent compounds.

Those skilled in the art will realize that a prodrug is a moiety that is generally not pharmacologically active. However, when activated, typically in vivo by enzymatic or hydrolytic cleavage to convert the prodrug to an active biological moiety, the administration of the prodrug to the individual will have had the intended medical effect. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

The phrase “protecting group” or “protective group” as used herein means a temporary substituent that protects a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

The term “residue” refers to that part of a compound that remains after the compound is linked, either directly to the other compound by a direct bond or to a divalent linking moiety. For instance, where a residue A₁ comprises a carboxylic acid group that forms a linkage to a second residue A₁ through an amino group to form the compound A₁-A₁, including an amide linkage, the first residue A₁ is the residue of the parent compound that includes all of the parent except for the —OH that forms part of the amide group, while the other includes all of the parent except an H− from the amino group. A person having skill in the art will recognize that this is analogous to “residues” of amino acids in polypeptides and proteins, or to “residues” of ribonucleotides and deoxyribonucleotides in RNA and DNA, respectively.

The term “substantially pyrogen-free” means a pharmaceutical composition having a pyrogen (e.g., endotoxin) concentration of less than about 0.3 EU/ml, preferably less than about 0.03 EU/ml, or even 0.01 EU/ml. The term also refers to a compound having a pyrogen contaminant (e.g., endotoxin) concentration of less than about 0.3 EU/mg, or even less than about 0.03 EU/mg, or possibly even less than about 0.01 EU/mg.

The phrase “within a patient” or “within the patient” refers, preferably, to that part of the patient that is below the patient's skin. In other embodiments, however, the phrase may refer to a patient's skin or to the exterior skin surface, as some embodiments allow the invention to be applied topically (for example, to treat a burn or laceration).

It will be apparent that various modifications, both to starting materials and methods, are encompassed by the invention and may be adopted without departing from the scope of the invention.

Furthermore, the foregoing examples are presented for illustrative purposes only, and are not intended to be limiting. The person skilled in the art will recognize that additional embodiments according to the invention are contemplated as being within the scope of the foregoing generic disclosure, and no disclaimer is in any way intended by the foregoing, non-limiting examples.

All patents, publications, and references cited in the foregoing disclosure are expressly incorporated herein by reference.

What is claimed is:

1. A drug delivery system for parenteral administration comprising:
(a) a pellet, wherein said pellet comprises at least one agent admixed with at least one super disintegrant compound, and
(b) an aqueous medium suitable for administering the at least one agent and at least one super disintegrant compound to a patient; and
wherein the pellet and aqueous medium are sterile and the at least one agent is a codrug comprising a non-steroidal anti-inflammatory drug and an opioid analgesic and the at least one disintegrant is selected from crospovidone, carmellose, directly compressible starches, modified starches, natural starches, and modified celluloses.

2. A drug delivery system for parenteral administration, comprising a pellet and an aqueous medium, the pellet having at least one agent in admixture with at least one disintegrant compound, wherein the pellet is dispersed in an aqueous medium to form a suspension suitable for administration to a patient; and the pellet and aqueous medium are sterile.

3. The drug delivery system according to claim 1, wherein the system is substantially pyrogen-free.

4. The drug delivery system of claim 1, wherein the at least one agent has a solubility of less than 1 mg/mL in the aqueous medium.

5. The drug delivery system of claim 1, having a release profile sufficient to deliver the at least one agent at a therapeutically effective dose over a period of at least 24 hours.

6. The drug delivery system of claim 1, having a release profile sufficient to deliver the at least one agent at a therapeutically effective dose over a period of at least 1 week.

7. The drug delivery system of claim 1, wherein the weight of the disintegrant compound is less than 10% of the weight of the pellet.

8. The drug delivery system of claim 2, wherein the pellet is dispersed in the aqueous medium within about 15 minutes of administering at least a portion of the suspension to a patient.

9. The drug delivery system of claim 1, wherein the pellet is less than 3 mm in diameter and less than 2 mm in height.

10. The drug delivery system of claim 1, wherein the pellet weighs less than 7 mg.

11. The drug delivery system of claim 1, wherein at least 90% of the pellet by weight is the at least one agent.

12. The drug delivery system of claim 1, wherein the at least one super disintegrant is sodium starch glycolate or croscarmellose sodium.

13. The drug delivery system of claim 1, wherein the at least one super disintegrant is croscarmellose sodium.

14. The drug delivery system of claim 1, wherein the pellet further includes at least one pharmaceutically acceptable carrier.

15. The drug delivery system of claim 14, wherein the pharmaceutically acceptable carrier is magnesium stearate.

16. The drug delivery system of claim 1, wherein the aqueous medium includes hyaluronic acid.

17. The drug delivery system of claim 1, wherein the aqueous medium includes saline solution.

18. The drug delivery system of claim 1, wherein the at least one agent has a decomposition half-life of less than about 1 month in aqueous solution at pH 7.4 and 25° C.

19. The drug delivery system of claim 1, wherein the at least one agent has a decomposition half-life of less than about 1 week in aqueous solution at pH 7.4 and 25° C.

20. The drug delivery system of claim 1, wherein the at least one agent has a decomposition half-life of less than about 24 hours in aqueous solution at pH 7.4 and 25° C.

21. The drug delivery system of claim 1, wherein the codrug includes a non-steroidal, anti-inflammatory drug covalently linked to an opioid analgesic.

22. The drug delivery system of claim 21, wherein the non-steroidal, anti-inflammatory drug is diclofenac or ketorolac and the opioid analgesic is morphine.

23. The drug delivery system of claim 1, wherein the disintegrant compound is a super disintegrant and is selected from one or more of carboxymethylcellulose, sodium starch glycolate, and cross-linked povidone.

24. The drug delivery system of claim 1, wherein the pellet and aqueous medium form a suspension when combined.

25. The drug delivery system of claim 1, wherein the pellet is greater than 1 mm and less than 10 mm in height.
